(19) United States
(12) Patent Application Publication
(10) Pub. No.: US 2022/0017845 A1
(43) Pub. Date: Jan. 20, 2022

(54) KIT FOR INSTALLING IMPELLER INTO PROCESS VESSEL

(71) Applicant: ENTEGRIS, INC., Billerica, MA (US)

(72) Inventors: Kurt CHRISTOFFERSEN, Millville, UT (US); Nicholas COSCIA, Lexington, MA (US); Gregory W. BORES, Prior Lake, MN (US)

(21) Appl. No.: 17/375,855

(22) Filed: Jul. 14, 2021

Related U.S. Application Data
(60) Provisional application No. 63/052,077, filed on Jul. 15, 2020.
(60) Provisional application No. 63/113,440, filed on Nov. 13, 2020.

(51) Int. Cl. C12M 1/00; B01F 7/00; B01F 7/18; B01F 15/00; C12M 1/06; C12M 3/00

(52) U.S. Cl. CPC C12M 23/14; B01F 7/0025; B01F 7/002; C12M 23/46; B01F 15/0085; C12M 27/02; B01F 7/18

(57) ABSTRACT

A kit for a bioreactor includes a bioreactor bag, a connector, an impeller assembly, and a clamp. The connector defines an aperture. The connector also includes a welding surface extending between a first end and a second end, with the second end including an outwardly extending flange. The welding surface can be heat-sealable to the bioreactor bag. The impeller assembly includes a shaft and a blade configured to pass through the aperture. The impeller assembly includes an impeller flange configured to contact the flange of the connector when the impeller shaft is received in the aperture. The impeller assembly also includes a bearing supporting the impeller shaft. The clamp is configured to secure the connector flange to the impeller assembly. Preparing a bioreactor includes welding the connector to the neck of the bioreactor bag and then inserting the shaft and blade through the aperture into the bioreactor bag.

### FIELD

This disclosure is directed to a kit for providing a process vessel with a single-use impeller assembly, in particular using a connector to be welded to the bag and clamped to the impeller assembly.

### BACKGROUND

Process vessels such as bioreactors can use single use bags to hold the reagents during a process. These bags can be welded directly to single use impeller devices to seal the process vessel to the impeller so that the impeller can stir the reagents. During the welding process, the impeller is already within the bag.

### SUMMARY

This disclosure is directed to a kit for providing a process vessel with a single-use impeller assembly, in particular using a connector to be welded to the bag and clamped to the impeller assembly.

Impeller assemblies for bioreactors can be large, requiring large tools to weld them to process vessels. Further, there may be space limitations making such welding difficult or less secure. In contrast, a connector can be welded to a process vessel using smaller welding tools and far more easily than a whole impeller assembly. The connector can provide an aperture allowing insertion of an impeller shaft into the process vessel. The impeller assembly can be secured to the connector by way of a clip, allowing the process vessel to be easily and simply connected to the impeller compared to direct welding. Welding the connector to the bag instead of the impeller also removes the complexity of finishing a three-dimensional bag with the impeller located within. Welding the connector to the bag instead of the impeller further removes the risk of impeller blades cutting or puncturing the bag during welding.

In an embodiment, a kit for a bioreactor includes a bioreactor bag including a neck, a connector defining an aperture and including a welding surface extending between a first end and a second end. The second end includes an outwardly extending flange. The welding surface is configured to be heat-sealed to the neck of the bioreactor bag. The kit further includes an impeller assembly. The impeller assembly includes an impeller shaft and impeller blade configured to pass through the aperture of the connector and an impeller flange configured to contact the outwardly extending flange of the connector when the impeller shaft is received in the aperture. The impeller assembly also includes a bearing supporting the impeller shaft. The kit further includes a clamp configured to secure the connector flange to the impeller assembly.

In an embodiment, the kit further includes a gasket configured to be disposed between the outwardly extending flange of the connector and the impeller flange. In an embodiment, the outwardly extending flange of the connector includes a channel configured to accommodate at least a portion of the gasket. In an embodiment, the impeller flange includes a channel configured to accommodate at least a portion of the gasket.

In an embodiment, at least one of the outwardly extending flange of the connector and the impeller flange includes a sealing feature.

In an embodiment, the bioreactor bag and the connector each include polyethylene. In an embodiment, the bioreactor bag and the connector each include a fluoropolymer.

In an embodiment, the connector is generally cylindrical.

In an embodiment, the welding surface is defined on an inner surface of the connector. In an embodiment, welding surface is defined on an outer surface of the connector.

In an embodiment, the neck of the bioreactor bag is joined to the welding surface of the connector by a weld.

In an embodiment, the bioreactor bag is a gusseted three dimensional bag.

In an embodiment, a method of assembling a bioreactor includes bonding a neck of a bioreactor bag to a welding surface of a connector. The connector defines an aperture and includes a connector flange. The connector flange is disposed outside the bioreactor bag. The method further includes inserting a blade and a shaft of an impeller assembly through the aperture of the connector into the bioreactor bag. The impeller assembly further includes an impeller flange configured to contact the connector flange when the blade and the shaft are inserted into the bioreactor. The method also includes clamping the connector flange to the impeller flange to secure the connector flange and the impeller flange to one another.

In an embodiment, the method further includes disposing a gasket between the impeller flange and the connector flange. In an embodiment, the gasket is disposed in at least one channel formed in one or more of the impeller flange and the connector flange.

In an embodiment, the bonding includes heat welding. In an embodiment, the heat welding is performed using a sealing port machine.

In an embodiment, a method includes assembling a bioreactor according to the methods discussed above, adding a bioreactor fluid to the bioreactor bag, and rotating the impeller blade within the bioreactor fluid.

In an embodiment, the bioreactor bag is a gusseted three dimensional bag.

In an embodiment, a kit for a bioreactor includes a bioreactor bag including a neck, a connector, and an impeller assembly. The connector defines an aperture and includes a welding surface surrounding the aperture. The welding surface is configured to be heat-sealed to the neck of the bioreactor bag. The impeller assembly includes an impeller shaft and impeller blade configured to pass through the aperture of the connector. The impeller assembly and the connector are configured to be mechanically joined to one another.

In an embodiment, the kit further includes a clamp, the connector includes an outwardly extending flange, and the impeller assembly includes an impeller flange configured to contact the outwardly extending flange of the connector. The impeller assembly and the connector are configured to be mechanically joined to one another by using the clamp to secure the outwardly extending flange of the connector and the impeller flange to one another.

In an embodiment the impeller assembly and the connector are configured to be mechanically joined to one another by a press-fit between the impeller assembly and a cup formed at an end of the connector.

In an embodiment, the impeller assembly and the connector are configured to be mechanically joined to one another by engagement features included on at least one of the impeller assembly or the connector.

In an embodiment, the impeller assembly and the connector are configured to be mechanically joined to one another by engagement of a captive nut disposed on one of the connector or the impeller assembly engaging with threading provided on the other of the connector or the impeller assembly.

In an embodiment, the impeller assembly and the connector are configured to be mechanically joined to one another by a plurality rotating bolts and locking nuts included in one of the impeller assembly or the connector, and a plurality of recesses configured to receive the rotating bolts formed in the other of the impeller assembly or the connector.

In an embodiment, when the impeller assembly and the connector are mechanically joined to one another, a seal is formed between the impeller assembly and the connector.

In an embodiment, the bioreactor bag and the connector each comprise polyethylene. In an embodiment, the bioreactor bag and the connector each comprise a fluoropolymer.

In an embodiment, the welding surface is defined on an inner surface of the connector. In an embodiment, the welding surface is defined on an outer surface of the connector.

In an embodiment, the bioreactor bag is a gusseted three dimensional bag.

In an embodiment, a method of assembling a bioreactor includes bonding a neck of a bioreactor bag to a welding surface of a connector. The connector defines an aperture. The method includes inserting a blade and a shaft of an impeller assembly through the aperture of the connector into the bioreactor bag. The method further includes mechanically fixing the impeller assembly to the connector.

In an embodiment, mechanically fixing the impeller assembly to the connector includes clamping an impeller flange included in the impeller assembly to a connector flange included in the connector.

In an embodiment, mechanically fixing the impeller assembly to the connector includes pressing the connector and the impeller assembly together to form a press-fit.

In an embodiment, mechanically fixing the impeller assembly to the connector includes bringing engagement features included on one of the impeller assembly or the connector into contact with the other of the impeller assembly or the connector.

In an embodiment, mechanically fixing the impeller assembly to the connector includes engaging a captive nut disposed on one of the impeller assembly or the connector with threading provided on the other of the impeller assembly or the connector.

In an embodiment, wherein the bonding includes heat welding. In an embodiment, the heat welding is performed using a sealing port machine.

In an embodiment, a method includes assembling a bioreactor, adding a bioreactor fluid to the bioreactor bag, and rotating the impeller blade within the bioreactor fluid.

In an embodiment, a kit for a process vessel includes a bag including a neck. The kit further includes a connector defining an aperture and including a welding surface surrounding the aperture, wherein the welding surface is configured to be heat-sealed to the neck of the bioreactor bag. The kit further includes an impeller assembly including an impeller shaft and impeller blade configured to pass through the aperture of the connector, wherein the impeller assembly and the connector are configured to be mechanically joined to one another.

In an embodiment, the kit further includes a clamp. The connector includes an outwardly extending flange, the impeller assembly includes an impeller flange configured to contact the outwardly extending flange of the connector, and the impeller assembly and the connector are configured to be mechanically joined to one another by using the clamp to secure the outwardly extending flange of the connector and the impeller flange to one another.

In an embodiment, the impeller assembly and the connector are configured to be mechanically joined to one another by a press-fit between the impeller assembly and a cup formed at an end of the connector.

In an embodiment, the impeller assembly and the connector are configured to be mechanically joined to one another by engagement features included on at least one of the impeller assembly or the connector.

In an embodiment, the impeller assembly and the connector are configured to be mechanically joined to one another by engagement of a captive nut disposed on one of the connector or the impeller assembly engaging with threading provided on the other of the connector or the impeller assembly.

In an embodiment, the impeller assembly and the connector are configured to be mechanically joined to one another by a plurality rotating bolts and locking nuts included in one of the impeller assembly or the connector, and a plurality of recesses configured to receive the rotating bolts formed in the other of the impeller assembly or the connector.

In an embodiment, when the impeller assembly and the connector are mechanically joined to one another, a seal is formed between the impeller assembly and the connector.

In an embodiment, the bag and the connector each comprise a polyolefin.

In an embodiment, the bag and the connector each comprise a fluoropolymer.

In an embodiment, the welding surface is defined on an inner surface of the connector.

In an embodiment, the welding surface is defined on an outer surface of the connector.

In an embodiment, the bag is a gusseted three dimensional bag.

In an embodiment, the process vessel is a bioreactor and the bag is a bioreactor bag.

In an embodiment, a method of assembling a process vessel including bonding a neck of a bag to a welding surface of a connector, the connector defining an aperture, inserting a blade and a shaft of an impeller assembly through the aperture of the connector into the bag, and mechanically fixing the impeller assembly to the connector.

In an embodiment, mechanically fixing the impeller assembly to the connector includes clamping an impeller flange included in the impeller assembly to a connector flange included in the connector.

In an embodiment, mechanically fixing the impeller assembly to the connector includes pressing the connector and the impeller assembly together to form a press-fit.

In an embodiment, mechanically fixing the impeller assembly to the connector includes bringing engagement features included on one of the impeller assembly or the connector into contact with the other of the impeller assembly or the connector.

In an embodiment, mechanically fixing the impeller assembly to the connector includes engaging a captive nut disposed on one of the impeller assembly or the connector with threading provided on the other of the impeller assembly or the connector.

In an embodiment, the bonding includes heat welding.

In an embodiment, the process vessel is a bioreactor and the bag is a bioreactor bag.

### DRAWINGS

FIG. 1 shows an exploded view of the components of a kit for a bioreactor according to an embodiment.

FIG. 2 shows a connector of a kit for a bioreactor according to an embodiment.

FIG. 3 shows a clamp of a kit for a bioreactor according to an embodiment.

FIG. 4 shows a sectional view of a connector and a portion of an impeller assembly according to an embodiment.

FIG. 5 shows a flowchart of a method of assembling a bioreactor.

FIG. 6 shows a connector and an impeller assembly according to an embodiment.

FIG. 7A shows a connector and an impeller assembly including a sectional view of a captive nut according to an embodiment.

FIG. 7B shows a connector and an impeller assembly including a sectional view of a captive nut according to an embodiment.

FIG. 8 shows a connector and an impeller assembly according to an embodiment.

### DETAILED DESCRIPTION

This disclosure is directed to a kit for providing a bioreactor bag with a single-use impeller assembly, in particular using a connector to be welded to the bag and clamped to the impeller assembly.

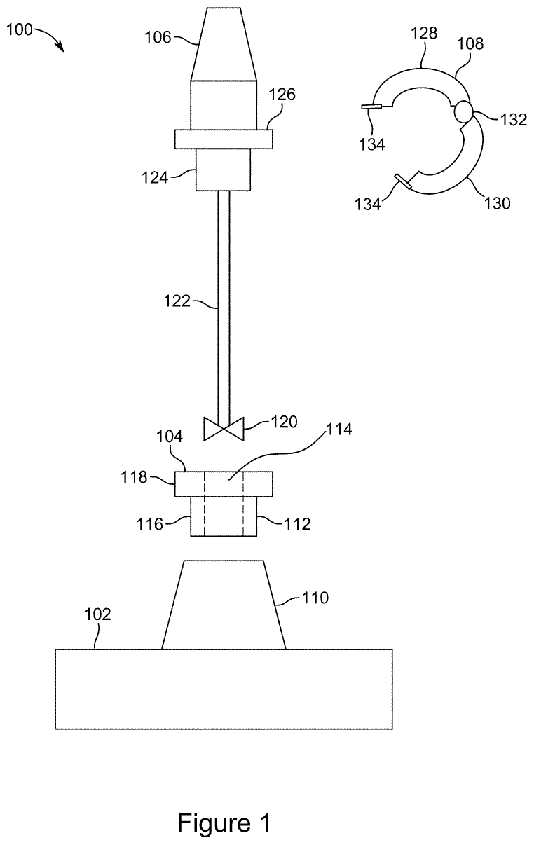

FIG. 1 shows an exploded view of the components of a kit for a bioreactor according to an embodiment. Bioreactor kit 100 includes a bag 102, a connector 104, an impeller assembly 106, and a clamp 108.

Bag 102 is a bioreactor bag configured to hold one or more reagents. Bag 102 can be any suitable material that will not adversely affect the reaction that will be performed therein. Bag 102 can be a single-use bag for storing process solutions such as chemical and/or biological reaction mixtures, reaction products, or the like. Bag 102 can be, for example, a bioprocess bag for storing a biological process solution. Bag 102 can be, for example, a part of a mixing system. In an embodiment, bag 102 can be, for example, a vessel used in or for a process such as a biological and/or chemical process. Bag 102 can be a flexible bag, for example, by incorporating one or more flexible materials in the bag. Bag 102 can have an operating temperature range suitable for the processes being conducted, which can include cryogenic processing or storage steps. The materials of bag 102 can be selected to reduce particulate generation, to reduce absorption of the contents of bag 102, and/or other characteristics improving purity and compatibility of bag 102 with process solutions to be stored therein. Bag 102 can be configured such that it can be sterilized, for example by gamma irradiation. Bag 102 can include one or more polymer materials, such as, for example, fluoropolymers and/or polyolefins, such as, as a non-limiting example, polyethylene. Bag 102 can have any suitable shape for containing reagents. Bag 102 can be, for example a two-dimensional or a three-dimensional bag. Bag 102 can be, for example, a gusseted three-dimensional bag. Bag 102 can be sized and/or shaped to fit within another vessel. Bag 102 can be any suitable size for a particular use, such as storage, mixing, use as a reaction vessel, or other suitable role in a chemical or biological process. In an embodiment, bag 102 is able to accommodate between 50 liters and 3000 liters.

Bag 102 includes a neck 110. Neck 110 is a portion of bag 102 that extends from bag 102. Neck 110 is shaped and sized to be sealed to the connector 104. Neck 110 is continuous with the internal space of bag 102. In an embodiment, bag 102 can include multiple necks 110. In this embodiment, the kit can include additional connectors 104, impeller assembly 106, and clamp, for example one of each for each neck 110 of the bag. In an embodiment, kit 100 may exclude the bag and include only a connector 104 and impeller assembly 106, and optionally further include clamp 108.

Connector 104 includes a connector body 112 defining an aperture 114. Connector body 112 includes a welding surface 116 formed on an inner surface, facing the aperture, or on an outer surface opposite the inner surface. Connector flange 118 is formed at one end of the connector body 112, extending outwards, away from the aperture 114.

Aperture 114 is an opening through connector 104 which is sized such that portions of impeller assembly 106 can pass through. In particular, aperture 114 is sized such that the impeller shaft 122 can be inserted through the aperture 114. In an embodiment, the impeller blades 120 can also be configured to pass through the aperture 114, for example by folding into a position such that the blades 120 can be passed through aperture 114.

Welding surface 116 is provided on a surface of connector body 112. The welding surface 116 is a portion of the connector body 112 that can be welded to the bag 102. The welding surface can include at least one material compatible with the material of bag 102 such that the connector 104 and the bag 102 can be joined by welding. The welding can be, for example, ultrasonic welding, heat welding, or any other suitable welding technique. In an embodiment, the bag 102 is joined to connector body 112 at welding surface 116 by a heat weld. In an embodiment, the weld can be provided by a sealing port machine. In an embodiment, welding surface 116 can instead be used to allow attachment of the bag 102 to the connector body 112 by any other suitable method of bonding the bag 102 to connector body 112, such as through use of an adhesive. For example, when the bag 102 is polyethylene, at least the welding surface 116 of connector 104 can also be polyethylene. The neck 110 of the bag 102 can be sized such that it can be sealed to the welding surface 116 by the weld joining the two. When the bag 102 is welded to the welding surface 116, the neck 110 of bag 102 is held open around aperture 114, such that the impeller shaft 122 and blades 120, when passed through the aperture 114, enters the interior space of bag 102.

Impeller assembly 106 is an assembly including impeller blades 120 located at an end of an impeller shaft 122. The impeller shaft 122 can be rotated to rotate the blades 120, for example to agitate reagents within bag 102 when a bioreactor is assembled from kit 100. The impeller assembly further includes a housing 124, which can contain a bearing (not shown) which allows rotation of shaft 122. Housing 124 includes an impeller flange 124.

Impeller blades 120 are blades configured to perturb a fluid when the impeller blades 120 are rotated. Impeller blades 120 can have any suitable geometry for agitating reagents within a process vessel such as bag 102. Impeller blades 120 can be configured such that they are capable of passing through aperture 114. In an embodiment, impeller blades 120 can be foldable into a position capable of passing through aperture 114 and have a deployed position incapable of being passed through aperture 114. In an embodiment, impeller blades 120 are sized such that they can be passed through the aperture 114.

Impeller shaft 122 is a shaft supporting impeller blades 120. Impeller shaft 122 is sized such that it can be passed through aperture 114, for example by having a diameter that is smaller than the diameter of the aperture 114. In an embodiment, impeller shaft 122 includes a motor interface (not shown) at an end opposite the impeller blades 120. The motor interface can be, for example, a gear or a mechanical engagement feature allowing the impeller shaft 122 to be driven to rotate.

Housing 124 includes at least a portion sized to fit within aperture 114. The housing 124 supports impeller shaft 122 by way of one or more bearings (not shown) contained within. Impeller flange 126 extends outwards from the housing 124. Impeller flange 126 extends outwards from housing 124 such that it cannot pass through aperture 114. Impeller flange 126 can be sized to match the dimensions of connector flange 118, for example having a same shape and size when viewed in cross-section. In an embodiment, the flange can include a channel (not shown) configured to accommodate a seal such as, for example, a gasket (not shown). In an embodiment, the channel can be configured to accommodate a sealing feature projecting from the opposing face of the connector flange 118.

Clamp 108 is a clamp configured to secure the connector flange 118 to the impeller flange 126. The clamp 108 can be any suitable clamp capable of securing the connector flange 118 to the impeller flange 126. In an embodiment, the clamp 108 includes a first segment 128 and a second segment 130, joined by hinge 132. The ends of first segment 128 and second segment 130 distal from the hinge 132 can be joined by securement 134. The first segment and second segment can each include a channel configured to accommodate the connector flange 118 and the impeller flange 126 when they are pressed against one another, with the channels surrounding at least portions of the connector flange 118 and impeller flange 126. The securement 134 can be, for example, a screw securement, a snap, or any other suitable mechanical connection for joining the respective ends of the first segment 128 and second segment 130. When the first segment 128 and the second segment 130 are joined by securement 134, the clamp 108 can surround the connector flange 118 and impeller flange 126 to retain them together.

In kit 100, the bag 102, connector 104, impeller assembly 106, and clamp 108 can be provided as a set of those individual components separate from one another. The elements of kit 100 can then be combined with one another to provide a bioreactor, for example by setting up bag 102 inside a containment vessel and filling it with the reagents, then welding connector 104 to bag 102, then inserting impeller assembly 106 through aperture 114 of connector 104, and finally clamping together the connector flange 118 and impeller flange 126 using the clamp 108. In an embodiment, two or more of those individual components can be pre-connected with one another, for example welding of the connector 104 to the bag 102 in the kit 100 prior to its assembly into a bioreactor.

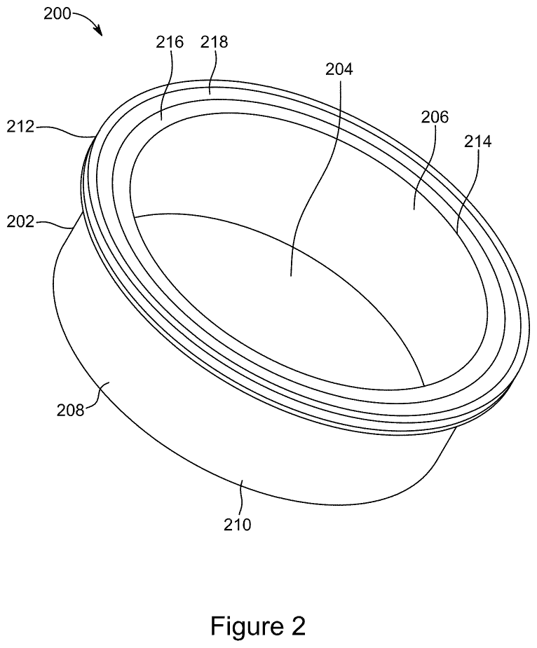

FIG. 2 shows a connector of a kit for a bioreactor according to an embodiment. Connector 200 includes a connector body 202 defining aperture 204. Connector body 202 includes inner surface 206 and outer surface 208. A connector flange 212 extends outwards from end 214 of the connector body 202. Connector flange 212 includes a seal feature 216.

Connector 200 is a connector configured to be connected to a process vessel, such as bag 102 described above and shown in FIG. 1, to facilitate insertion of an impeller assembly such as impeller assembly 106 into the bag and to secure the impeller assembly and bag together. Connector 200 can be more readily assembled with and welded to the process vessel such as a bioreactor bag, when compared to an entire impeller assembly.

Connector body 202 forms the connector 200. Connector body 202 can have any suitable shape for being joined to the bag and to provide an aperture through which a shaft of In an embodiment, the connector body 202 has a generally cylindrical shape. The connector body 202 can have a rounded cross-sectional shape such as a circular or oval cross-sectional shape. In an embodiment, the connector body 202 has a cross-sectional shape selected for welding to a neck portion of a bag such as bag 102. The connector body 202 can be any suitable material for contacting a bag such as bag 102 or any reagents possibly contained therein. The connector body 202 can include, for example, polymer materials such as polyethylene or fluoropolymers. In an embodiment, the connector body 202 is made entirely of polyethylene or a fluoropolymer.

Aperture 204 is an opening defined by connector body 202. Aperture 204 is sized such that an impeller shaft and blades can be passed through the aperture from one side of the connector body 202 to the opposite side of connector body 202.

Inner surface 206 is a surface of connector body 202 facing inwards towards aperture 204. Outer surface 208 is a surface of connector body 202 opposite the inner surface 206, facing outwards away from aperture 204. Welding surface 210 can be provided on one of inner surface 206 or outer surface 208. The welding surface 210 is a surface capable of being welded to a bag, for example capable of being heat-welded to the bag.

Connector flange 212 extends outwards from connector body 202, away from aperture 204, at end 214 of the connector body 202. Connector flange 212 can be continuous surrounding aperture 204. Connector flange 212 can maintain the general cross-sectional shape of the connector body 202, for example continuing to be circular when the cross-section of connector body 202 is circular, or oval or oval when the cross-section of connector body 202 is oval. In an embodiment, the shape and dimensions of connector flange 212 are selected to correspond to the shape and dimensions of a flange provided on an impeller assembly to be used with the connector 200.

Seal feature 216 can be provided on a flange surface 218 of connector flange 212. Seal feature 216 can surround aperture 204. In an embodiment, the seal feature 216 is a projection or ridge surrounding aperture 204. In an embodiment, the seal feature 216 is a gasket disposed in a channel formed in the flange surface 218. In an embodiment, the gasket is a resilient material and sized to be compressed when the connector flange 212 is secured to a flange of an impeller assembly, for example when the flanges are clamped together. The gasket can be, for example, a polymer material. In an embodiment, the gasket is silicone.

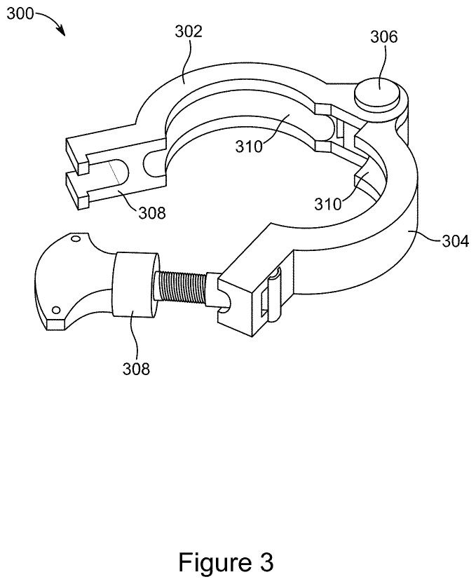

FIG. 3 shows a clamp of a kit for a bioreactor according to an embodiment. Clamp 300 includes a first segment 302, joined to second segment 304 by a hinge 306, with securement 308 provided opposite hinge 306 when the clamp 300 is closed. Clamp 300 includes channel 310 formed in the inward-facing surfaces of first segment 302 and second segment 304.

Clamp 300 can be used to clamp together a flange provided on an impeller assembly and a corresponding flange provided on a connector. The connector can be welded to a bag to which the impeller assembly will be joined, such as a bioreactor. In an embodiment, clamp 300 is a tri-clamp. In an embodiment, clamp 300 has a threaded screw closure.

First segment 302 and second segment 304 are separate segments that, when joined together by hinge 306 and securement 308, form a continuous shape configured to surround flanges of an impeller assembly and a connector. The continuous shape can correspond to the cross-sectional shapes of the flanges of the impeller assembly and the connector. In an embodiment, first segment 302 and second segment 304 define a shape having a circular cross-sectional shape. In an embodiment, each of first segment and second segment are of approximately the same length, each defining approximately one half of the clamp 300.

Hinge 306 joins first segment 302 to second segment 304. Hinge 306 can be any suitable hinge allowing relative movement of first and second segments 302 and 304 with respect to one another by rotation about the hinge 306. In an embodiment, hinge 306 is formed by a pin passing through holes provided on each of first and second segments 302 and 304. In an embodiment, hinge 306 is formed by the mechanical fit of portions of first and second segments 302 and 304 having detents or other features that can be rotated about.

Securement 308 joins the ends of first segment 302 and second segment 304 that are distal from hinge 306 to one another. Securement 308 can use any suitable mechanical means of joining first segment 302 to second segment 304. In an embodiment, securement 308 includes engagement features provided on first and second segments 302 and 304 that interface to form a snap fit. In an embodiment, one of first segment 302 and second segment 304 includes a swingable screw 312, and the other of first segment 302 and second segment 304 includes an engagement feature 316 configured to receive the swingable screw. A bolt 314 can be provided on the swingable screw 312 such that it can be rotated into a position where it presses against the engagement feature 316 to secure the first segment to the second segment, particularly when clamp 300 surrounds flanges of an impeller assembly and a connector.

Channel 310 is a channel provided on an inwards-facing surface of each of first segment 302 and second segment 304. The channel 310 is sized to accommodate the outer edges of flanges provided on each of a connector and an impeller assembly. Channel 310 is configured to secure the flange on the connector to the flange on the impeller assembly such that their respective facing surfaces are pressed together. In an embodiment, the pressing of the facing surfaces of the flanges can compress a gasket to form a seal around an aperture formed in the connector.

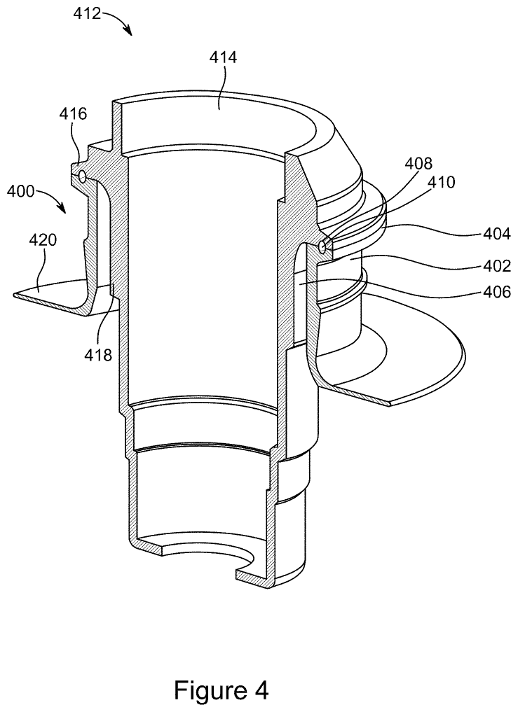

FIG. 4 shows a sectional view of a connector and a portion of an impeller assembly according to an embodiment. Connector 400 includes connector body 402, with connector flange 404 extending outwards. Connector 400 defines an aperture 406. A channel 408 is provided on a surface of flange 404. The channel 408 accommodates gasket 410. Impeller assembly 412 includes housing 414, which includes housing flange 416 extending outwards. Housing 414 includes an extension 418 extending through aperture 406.

Connector 400 is configured to be welded to a bag, for example using a weld such as a heat weld. The connector 400 is configured to allow a shaft and blades of an impeller assembly 412 to pass through into an interior of the bag to which the connector 400 is welded. Connector 400 can be provided along with the impeller assembly 412, a clamp, and optionally also with a bag such as a bioreactor bag to provide a kit for assembling a process vessel.

Connector body 402 includes a welding surface and connector flange 404. Connector flange 404 is a flange extending outwards from an end of the connector body. Connector flange 404 can be sized and shaped to correspond to housing flange 416 such that the flanges can be clamped to one another. Connector body 402 further is shaped to define aperture 406.

Aperture 406 is an opening through connector 400 that is defined by the connector body 402. Aperture 406 is sized to allow a shaft and blades of impeller assembly 412 to pass through the connector body 402. When the connector 400 is welded to a bag, aperture 406 allows an impeller to pass from an exterior of the bag to the internal space of the bag. Aperture 406 can further also accommodate extension 418 of housing 414 of the impeller assembly. Aperture 406 is sized such that the housing flange 416 cannot pass through the aperture 406.

Sealing channel 408 is a channel formed in the surface of connector flange 404 surrounding aperture 406. Sealing channel 408 can be shaped and sized to accommodate a portion of seal 410, such that seal 410 can provide a seal between the connector flange 404 and the housing flange 416, surrounding aperture 406.

Seal 410 can be a gasket, O-ring, or any other suitable sealing member. The seal 410 in the embodiment shown in FIG. 4 is a gasket. The seal 410 can extend above the surface of connector flange 404, out of the sealing channel 408. The seal 410 can include a resilient material. The seal 410 can be compressed between the connector flange 404 and the housing flange 416. The seal 410 can be at least partially disposed within sealing channel 408. In an embodiment, another portion of the seal 410 can be received in a channel formed in a surface of housing flange 416. The seal 410 can be a polymer material. In an embodiment, the seal 410 is made of silicone.

In the embodiment shown in FIG. 4, the connector 400 includes a welding surface flange 420. The welding surface flange extends outwards from the end of the connector 400 opposite the connector flange 404. The welding surface flange 420 can include the welding surface configured to be welded to the bag. The bag and the welding surface flange 420 can be sized relative to one another such that the bag can be placed in contact with the welding surface provided on the welding surface flange 420.

Impeller assembly 412 includes housing 414. Impeller assembly 412 can further include a shaft (not shown), passing through housing 414 and supported by bearings (not shown). Blades (not shown) can be located at an end of the shaft. The housing 414 is a portion of impeller assembly 412 configured to be fixed to the connector 400, for example by clamping, while allowing the shaft to rotate freely, for example by supporting the shaft by way of the bearings.

Housing flange 416 is a projection outwards from the housing 414. The housing flange 416 can have a size and shape that correspond at least generally to the size and shape of connector flange 404, such that the housing flange 416 and connector flange 404 can be clamped together. In an embodiment, a sealing channel similar to sealing channel 408 can be provided on the surface of housing flange 416 facing the connector flange 404.

Extension 418 is a portion of housing 414 sized such that it can fit within aperture 406. The extension 418 can provide separation between a shaft (not shown) of the impeller assembly and the connector body 402. The extension 418 can extend at least a portion of the length of aperture 406 through connector body 402. In an embodiment, extension 418 extends all the way through aperture 406 beyond the end of connector body 402.

While FIG. 4 shows the connector 400 and the impeller assembly 412 connected to one another, this is to show the relative sizing and shaping of the connector and impeller assembly. Kits according to embodiments can include the connector 400 and the impeller assembly 412 as separate components configured to be subsequently assembled together, to result in the combination of connector 400 and impeller assembly 412 shown in FIG. 4. Embodiments can also include methods of combining the connector 400 and impeller assembly 412 to result in the arrangement shown in FIG. 4.

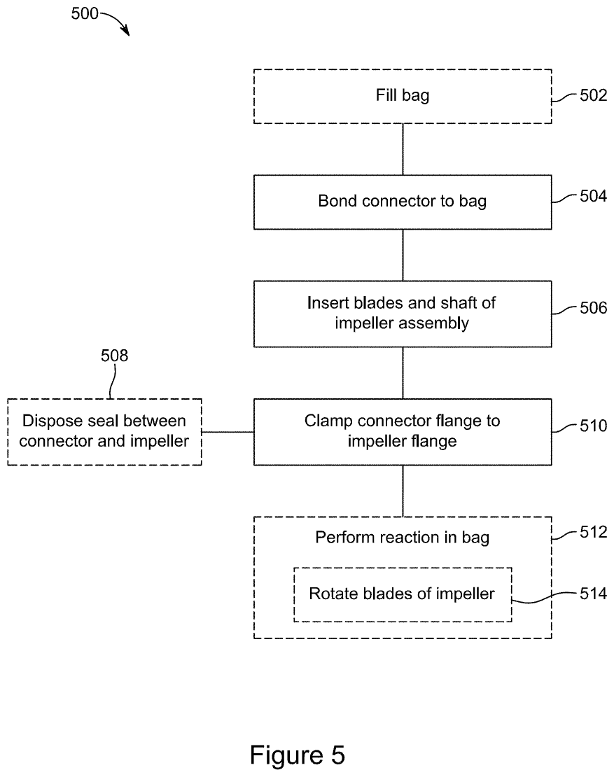

FIG. 5 shows a flowchart of a method of assembling a bioreactor. In method 500, a bag is optionally filled with reagents 502. A connector is bonded to a neck of the bag 504. One or more blades and a shaft of an impeller assembly are inserted into the bag through an aperture in the connector 506. Optionally, a seal can be disposed between the impeller assembly and the connector 508. The impeller assembly is clamped to the connector 510. Optionally, the bag can be used to conduct a reaction 512, which can optionally include rotating the shaft and blades of the impeller assembly 514.

A bag is optionally filled with reagents 502. The bag can be, for example, bag 102 shown in FIG. 1 and described above. The filling at 502 can be accomplished through either a neck formed in the bag or a separate filling port of the bag. The reagents can be any reagents used in the process the bag is to provide a process vessel for. The bag can be filled at 502 prior to bonding of the connector to the bag at 504. In an embodiment, the bag can be filled at 502 subsequent to bonding of the connector to the bag at 504. In an embodiment, the bag can be filled at 502 subsequent to clamping of the impeller assembly to the connector at 510. In this embodiment, the bag is filled 502 using a fill port separate from the neck of the bag.

A connector is bonded to a neck of the bag 504. The connector can be, for example, connector 104 or connector 200 described above and shown in FIGS. 1 and 2, respectively. The bag can be, for example, bag 102 having neck 110 as described above and shown in FIG. 1. The neck of the bag can be placed in proximity to or contact with a welding surface on the connector, and the neck and welding surface then are bonded. The bonding can be done by way of heat welding to join the neck and welding surface. The bonding can be performed using, for example, a sealing port machine. The bonding of the welding surface of the connector and the neck of the bag at 504 can be performed before or after filling of the bag 502. In an embodiment, the connector can be bonded to the neck of the 504 prior to installation of the bag into a containment vessel.

One or more blades and a shaft of an impeller assembly are inserted into the bag through an aperture in the connector 506. In an embodiment, the blades of the impeller assembly can be placed into a folded position such that they can pass through the aperture of the connector. The insertion of the blades and shaft into be bag at 506 can be performed after the joining of the connector to the bag at 504. The impeller assembly may be moved into the aperture of the connector until a flange of the impeller assembly, which is too large to pass through the aperture, contacts a flange of the connector.

Optionally, a seal can be disposed between the impeller assembly and the connector 508. The seal surrounds the aperture of the connector. The seal can be disposed between the impeller assembly and the connector 508 at any time before clamping of the impeller assembly to the connector at 510. In an embodiment, the seal can include one or more features such as ridges, channels, overmolded portions, or the like on one or both of the flange of the connector or the flange of the impeller assembly. In an embodiment, the seal can be a gasket, O-ring, or other sealing member separate from the connector and the impeller assembly. The gasket, O-ring, or other sealing member can be received in one or more channels provided in one or both of the flange of the connector and the flange of the impeller assembly. In an embodiment, the gasket, O-ring or other sealing member can be placed between the flanges of the connector and the impeller assembly, and compressed when the flanges are clamped together at 510. The seal can include, for example, a polymer material. The seal can include, for example, silicone.

The impeller assembly is clamped to the connector 510. The clamping can be clamping of the flange of the impeller assembly to the flange of the connector, using a clamp such as clamp 108 or clamp 300 described above and shown in FIGS. 1 and 3, respectively. The clamping includes placing one or more clamps onto the flanges of the impeller assembly and the connector when those flanges are in close proximity or in contact with one another. The clamping can include rotating segments of the clamp about a hinge to surround the flanges of the impeller assembly and the connector. The clamping can include securing closure of the clamp using a securement such as a snap, a screw, or any other suitable mechanical closure of the clamp. The clamping of the impeller assembly to the connector at 510 can apply force pressing the respective flanges of the impeller assembly and connector towards one another. The force can contribute to the sealing of the joint between the impeller assembly and the flange around the aperture, for example by pressing together sealing features, or compressing a gasket, O-ring, or other sealing member.

Optionally, the bag can then be used to conduct a reaction 512, which can optionally include rotating the shaft and blades of the impeller assembly 514. The reaction can be any suitable reaction of the reagents contained within the bag. The reaction of 512 can be facilitated by agitation, for example by the rotation of the shaft and blades of the impeller assembly at 514. The shaft and blades of the impeller assembly can be driven to perform the rotation at 514 by a mechanical interface with a motor, such as interface by way of gears, using a socket provided on an end of the shaft opposite the blades, or any other suitable mechanical linkage for driving rotation of the shaft. The shaft can be retained while being allowed to rotate by one or more bearings included in a housing of the impeller assembly, with the housing being the part including the flange clamped to the connector at 510.

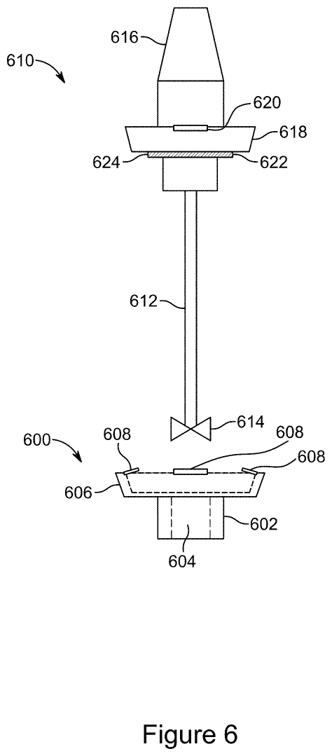

FIG. 6 shows a connector and an impeller assembly according to an embodiment. Connector 600 and impeller assembly 610 are configured to be joined to one another to form a sealed connection between the impeller assembly 610 and an interior of a reactor bag (not shown) that is joined to the connector 600.

Connector 600 includes a connector body 602 and an aperture 604 passing through the connector body 602. A cup 606 configured to receive a connector interface portion 618 of impeller assembly 610 can be disposed at one end of connector body 602, with one end of the aperture 604 positioned within cup 606. The cup 606 can optionally include one or more connector engagement features 608. Connector engagement features 608 can be any suitable feature for interfacing with connector interface portion 618 or impeller assembly engagement features 620 to retain the connector interface portion 618 within cup 606. Non-limiting examples of connector engagement features 608 include tabs configured to provide a snap fit with connector interface portion 618, depressions, slots, or channels configured to receive projections from a surface of connector interface portion 618, detents, projections, or any other suitable feature for securing the connector interface portion 618 within cup 606.

Impeller assembly 610 includes a shaft 612, a blade 614, and motor housing 616. Motor housing 616 includes a connector interface portion 618 configured to be received in cup 606 of connector 600 when shaft 612 is extending through aperture 604 of the connector 600. The connector interface portion 618 can be configured to be retained within cup 606 when shaft 612 is extending through aperture 604. As a non-limiting example, connector interface portion 618 can be sized and/or shaped to form a press-fit with cup 606. Motor housing 616 can include one or more impeller assembly engagement features 620 to allow the retention of connector interface portion 618 within cup 606. The impeller assembly engagement features 620 can be provided, for example, on the connector interface portion 618 of motor housing 616. Impeller assembly engagement features 620 can be, as non-limiting examples, tabs or projections configured to engage with connector engagement features 608 to provide a snap fit, projections configured to be received in channels or slots when those are provided as connector engagement features 608, or any other suitable feature for forming a connection between motor housing 616 and cup 606 that is capable of retaining the connector interface portion 618 within the cup 606 while shaft 612 is extending through aperture 604.

In an embodiment, a seal 622 can be included. In an embodiment, the seal 622 can surround aperture 604. In an embodiment such as that shown in FIG. 6, seal 622 can surround where shaft 612 extends from the connector interface portion 618. In an embodiment, the seal 622 can surround a surface of connector interface portion 618 contacting cup 606. In an embodiment, the seal 622 can be disposed on an interior surface of cup 606 that is configured to contact connector interface portion 618. In an embodiment, the seal 622. In embodiments, multiple seals 622 can be included, for example with the seals being concentric 0-rings or gaskets. In embodiments with multiple seals 622, the different seals 622 can each be located in different suitable locations for a seal 622, for example with one disposed on an interior surface of cup 606 and another disposed on connector interface portion 618. The seal 622 can be disposed in a channel 624 which can be formed in the cup 606 or connector interface portion 618 depending on which part the seal 622 is disposed on. In the embodiment shown in FIG. 6, the channel 624 is formed in connector interface portion 618.

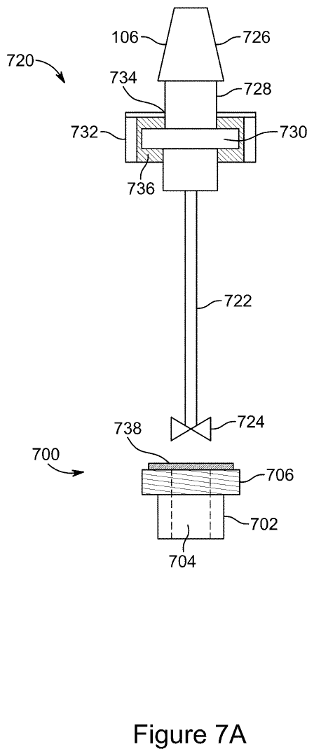

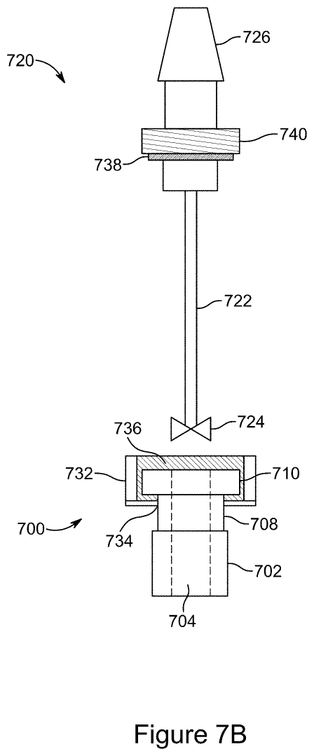

FIG. 7A shows a connector and an impeller assembly including a sectional view of a captive nut according to an embodiment. Connector 700 and impeller assembly 720 are configured to be joined to one another to form a sealed connection between the impeller assembly and an interior of a reactor bag (not shown) that is joined to the connector 700.

Connector 700 includes connector body 702 and an aperture 704 passing through the connector body 702. Connector body 702 includes a threaded extension 706 at one end. Threaded extension 706 is sized to engage with threading 736 of captive nut 732, such that the captive nut can be used to secure the impeller assembly 720 to the connector 700.

Impeller assembly 720 includes shaft 722, blade 724, and motor housing 726. Motor housing 726 includes a neck 728 and an extension 730. Captive nut 732 surrounds neck 728 and extension 730. Captive nut 732 includes an aperture 734 at one end, with aperture 734 sized such that captive nut 732 can be moved along neck 728 of the motor housing 726, but cannot pass the extension 730. Captive nut 732 includes an internal space configured to surround extension 730 and to extend past it when captive nut 732 is at a point where neck 728 meets extension 730. The internal surface of captive nut 732 defining this internal space includes threading 736. Threading 736 is configured to engage with the threaded extension 706 of connector 700. When captive nut 732 is tightened to threaded extension 706, the contact between captive nut 732 and impeller assembly 720 can retain impeller assembly 720 to connector 700.

In an embodiment, a seal 738 can be provided to seal the connection between the impeller assembly 700 and connector 700. As non-limiting examples, seal 738 can be provided on threaded extension 706, within captive nut 732, surrounding aperture 704, on extension 730, for example where it contacts connector 700 when impeller assembly 720 is retained to connector 700, or any other such suitable position to provide a seal.

FIG. 7B shows a connector and an impeller assembly including a sectional view of a captive nut according to an embodiment. In the embodiment shown in FIG. 7B, connector body 702 includes a neck 708 and a flange 710. The captive nut 732 is configured to surround neck 708 and aperture 734 is sized such that captive nut 732 can move along neck 708 but cannot pass flange 710. In an embodiment, seal 738 is disposed on flange 710. In an embodiment, channel 712 is formed on flange 710 and configured to accommodate seal 738. Seal 738 is sized and positioned such that it can form a seal surrounding aperture 704 when the impeller assembly 720 is secured to connector 700 using captive nut 732.

In the embodiment shown in FIG. 7B, motor housing 726 includes threading 740 configured to engage the threading 736 of the captive nut 732. In the embodiment shown in FIG. 7B, when captive nut 732 is tightened to motor housing 726 by the engagement of the respective threading 736, 740, contact between captive nut 732 and flange 710 retains connector 700 and impeller assembly 720 together. In an embodiment, seal 738 contacts motor housing 726 and forms a seal surrounding aperture 704 of the connector 700.

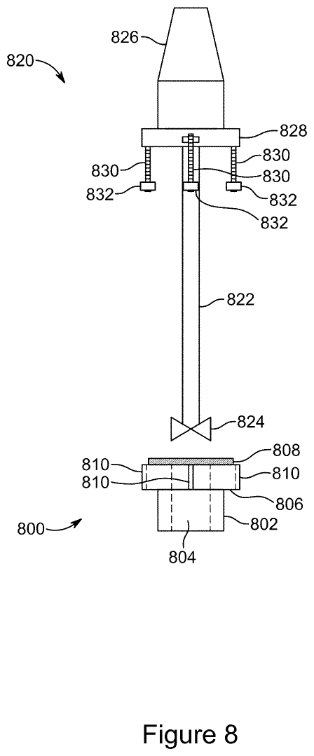

FIG. 8 shows a connector and an impeller assembly according to an embodiment. Connector 800 and impeller assembly 820 are configured to be joined to one another to form a sealed connection between the impeller assembly 820 and an interior of a reactor bag (not shown) that is joined to the connector 800.

Connector 800 includes a connector body 802, an aperture 804 passing through the connector body 802, a flange 806 surrounding the aperture, and a seal 808 disposed in the flange 806. Connector body 802 defines the connector. Aperture 804 is an aperture sized to allow the shaft 824 of impeller assembly 820 to pass through, such that the shaft 824 can enter a reactor bag bonded to the connector 800. The flange 806 can include a plurality of recesses 810 configured to each receive a movable bolt 830, with portions of flange 806 surrounding the recesses 810 being configured to contact a locking nut 832 when the locking nut 832 is tightened. In the embodiment shown in FIG. 8, seal 808 is provided on flange 806. Seal 808 is a seal configured to contact housing flange 828 of the impeller assembly 820 when impeller assembly 820 is joined to connector 800. In an embodiment, the seal 808 is disposed in a channel 812 formed in the flange 806. In embodiments, seal 808 can instead or additionally be disposed on the housing flange 828 of the impeller assembly 820 and configured to contact the flange 806 when the impeller assembly 820 is joined to connector 800. The seal 808 can be any suitable seal capable of forming a seal around the aperture 804 when the impeller assembly 820 is joined to connector 800. As one non-limiting example, the seal 808 is a flat gasket.

Impeller assembly 820 includes a motor housing 822, a shaft 824, and one or more impeller blades 826. The motor housing 822 can include a housing flange 828. Housing flange 828 is sized such that it cannot pass through aperture 804 of the connector 800. The housing flange 828 can include a plurality of movable bolts 830. The movable bolts 830 can be pivotally connected to the housing flange 828. The movable bolts 830 can be distributed around housing flange 828 such that they correspond to recesses 814 of connector 800 when the impeller assembly 820 is to be joined to the connector 800. The movable bolts 830 can be threaded, with locking nuts 832 engaged with the threading of movable bolts 830. The locking nuts 832 can be configured to move up and down movable bolts 830 by rotation of the locking nuts 832. The locking nuts 832 and movable bolts 830 can be configures such that when the movable bolts 830 are rotated into the recesses 814 of flange 806, the locking nuts 832 can be rotated such that they contact flange 806 such that they apply pressure holding flange 806 and housing flange 828 together, joining connector 800 to impeller assembly 820. The pressure provided by contact of locking nuts 832 and flange 806 can compress seal 816.

In an embodiment, movable bolts 830 can be rotatably attached to flange 806 of the connector 800, and recesses 814 can instead be provided on housing flange 828. In this embodiment, the portions of housing flange 828 surrounding recesses 814 can be configured to be contacted by the locking nuts 832, with pressure applied to housing flange 828 by the locking nuts 832 holding the housing flange 828 to the flange 806.

Embodiments can include any other suitable mechanical joining of the impeller assemblies to connectors that are capable of providing sealing between the impeller assembly and the passage through the connector into the bag, in addition to the connections using clamps or other mechanical connections such as those particularly detailed herein and shown in the respective Figures.

Embodiments can further be applied to other chemical reactors in addition to bioreactors, and can be used to contain and stir other reagent solutions such as for chemical processes. In an embodiment, a kit according to an embodiment or assembled according to an embodiment can contain a mixture of liquids or a mixture of liquids and powders that are reagents for a chemical reaction. The chemical reaction can be a chemical reaction performed under stirring by the impeller assembly, for example gentle stirring to mix the reagents.

### ASPECTS

It is understood that any of aspects 1-13 can be combined with any of aspects 13-19, 20-32, 33-39, 40-52, or 53-59. It is understood that any of aspects 13-19 can be combined with any of aspects 20-32, 33-39, 40-52, or 53-59. It is understood that any of aspects 20-32 can be combined with any of aspects 33-39, 40-52, or 53-59. It is understood that any of aspects 33-39 can be combined with any of aspects 40-52, or 53-59. It is understood that any of aspects 40-52 can be combined with any of aspects 53-59.

Aspect 1. A kit for a bioreactor, comprising:

a bioreactor bag including a neck;
a connector defining an aperture and including a welding surface extending between a first end and a second end, the second end including an outwardly extending flange, the welding surface configured to be heat-sealed to the neck of the bioreactor bag;
an impeller assembly including an impeller shaft and impeller blade configured to pass through the aperture of the connector, an impeller flange configured to contact the outwardly extending flange of the connector when the impeller shaft is received in the aperture, and a bearing supporting the impeller shaft; and
a clamp configured to secure the connector flange to the impeller assembly.

Aspect 2. The kit according to aspect 1, further comprising a gasket configured to be disposed between the outwardly extending flange of the connector and the impeller flange.

Aspect 3. The kit according to aspect 2, wherein the outwardly extending flange of the connector includes a channel configured to accommodate at least a portion of the gasket.

Aspect 4. The kit according to any of aspects 2 or 3, wherein the impeller flange includes a channel configured to accommodate at least a portion of the gasket.

Aspect 5. The kit according to any of aspects 1-4, wherein at least one of the outwardly extending flange of the connector and the impeller flange includes a sealing feature.

Aspect 6. The kit according to any of aspects 1-5, wherein the bioreactor bag and the connector each comprise polyethylene.

Aspect 7. The kit according to any of aspects 1-6, wherein the bioreactor bag and the connector each comprise a fluoropolymer.

Aspect 8. The kit according to any of aspects 1-7, wherein the connector is generally cylindrical.

Aspect 9. The kit according to any of aspects 1-8, wherein the welding surface is defined on an inner surface of the connector.

Aspect 10. The kit according to any of aspects 1-8, wherein the welding surface is defined on an outer surface of the connector.

Aspect 11. The kit according to any of aspects 1-10, wherein the neck of the bioreactor bag is joined to the welding surface of the connector by a weld.

Aspect 12. The kit according to any of aspects 1-11, wherein the bioreactor bag is a gusseted three dimensional bag.

Aspect 13. A method of assembling a bioreactor, comprising:

bonding a neck of a bioreactor bag to a welding surface of a connector, the connector defining an aperture and including a connector flange, the connector flange disposed outside the bioreactor bag;
inserting a blade and a shaft of an impeller assembly through the aperture of the connector into the bioreactor bag, the impeller assembly further including an impeller flange configured to contact the connector flange when the blade and the shaft are inserted into the bioreactor; and
clamping the connector flange to the impeller flange to secure the connector flange and the impeller flange to one another.

Aspect 14. The method according to aspect 13, further comprising disposing a gasket between the impeller flange and the connector flange.

Aspect 15. The method according to aspect 14, wherein the gasket is disposed in at least one channel formed in one or more of the impeller flange and the connector flange.

Aspect 16. The method according to any of aspects 13-15, wherein the bonding includes heat welding.

Aspect 17. The method according to aspect 16, wherein the heat welding is performed using a sealing port machine.

Aspect 18. A method comprising:

assembling a bioreactor according to the method of any of aspects 13-17,
adding a bioreactor fluid to the bioreactor bag; and
rotating the impeller blade within the bioreactor fluid.

Aspect 19. The method according to any of aspects 13-18, wherein the bioreactor bag is a gusseted three dimensional bag.

Aspect 20. A kit for a bioreactor, comprising:

a bioreactor bag including a neck;
a connector defining an aperture and including a welding surface surrounding the aperture, wherein the welding surface is configured to be heat-sealed to the neck of the bioreactor bag; and
an impeller assembly including an impeller shaft and impeller blade configured to pass through the aperture of the connector,
wherein the impeller assembly and the connector are configured to be mechanically joined to one another.

Aspect 21. The kit of claim 20, further comprising a clamp, wherein:

the connector includes an outwardly extending flange,
the impeller assembly includes an impeller flange configured to contact the outwardly extending flange of the connector, and
the impeller assembly and the connector are configured to be mechanically joined to one another by using the clamp to secure the outwardly extending flange of the connector and the impeller flange to one another.

Aspect 22. The according to aspect 20 or aspect 21, wherein the impeller assembly and the connector are configured to be mechanically joined to one another by a press-fit between the impeller assembly and a cup formed at an end of the connector.

Aspect 23. The kit according to any of aspects 20-22 wherein the impeller assembly and the connector are configured to be mechanically joined to one another by engagement features included on at least one of the impeller assembly or the connector.

Aspect 24. The kit according to any of 20-23, wherein the impeller assembly and the connector are configured to be mechanically joined to one another by engagement of a captive nut disposed on one of the connector or the impeller assembly engaging with threading provided on the other of the connector or the impeller assembly.

Aspect 25. The kit according to any of aspects 20-24, wherein the impeller assembly and the connector are configured to be mechanically joined to one another by a plurality rotating bolts and locking nuts included in one of the impeller assembly or the connector, and a plurality of recesses configured to receive the rotating bolts formed in the other of the impeller assembly or the connector.

Aspect 26. The kit according to any of aspects 20-25, wherein when the impeller assembly and the connector are mechanically joined to one another, a seal is formed between the impeller assembly and the connector.

Aspect 27. The kit according to any of aspects 20-26, wherein the bioreactor bag and the connector each comprise polyethylene.

Aspect 28. The kit according to any of aspects 20-27, wherein the bioreactor bag and the connector each comprise a fluoropolymer.

Aspect 29. The kit according to any of aspects 20-28, wherein the welding surface is defined on an inner surface of the connector.

Aspect 30. The kit according to any of aspects 20-29, wherein the welding surface is defined on an outer surface of the connector.

Aspect 31. The kit according to any of aspects 20-30, wherein the bioreactor bag is a gusseted three dimensional bag.

Aspect 32. A method of assembling a bioreactor, comprising:

bonding a neck of a bioreactor bag to a welding surface of a connector, the connector defining an aperture;
inserting a blade and a shaft of an impeller assembly through the aperture of the connector into the bioreactor bag; and
mechanically fixing the impeller assembly to the connector.

Aspect 33. The method according to aspect 32, wherein mechanically fixing the impeller assembly to the connector includes clamping an impeller flange included in the impeller assembly to a connector flange included in the connector.

Aspect 34. The method according to aspect 32 or aspect 33, wherein mechanically fixing the impeller assembly to the connector includes pressing the connector and the impeller assembly together to form a press-fit.

Aspect 35. The method according to any of aspects 32-34, wherein mechanically fixing the impeller assembly to the connector includes bringing engagement features included on one of the impeller assembly or the connector into contact with the other of the impeller assembly or the connector.

Aspect 36. The method according to any of aspects 32-35, wherein mechanically fixing the impeller assembly to the connector includes engaging a captive nut disposed on one of the impeller assembly or the connector with threading provided on the other of the impeller assembly or the connector.

Aspect 37. The method according to any of aspects 32-36, wherein the bonding includes heat welding.

Aspect 38. The method according to aspect 37, wherein the heat welding is performed using a sealing port machine.

Aspect 39. A method comprising:

assembling a bioreactor according to the method according to any of aspects 31-37,
adding a bioreactor fluid to the bioreactor bag; and
rotating the impeller blade within the bioreactor fluid.

Aspect 40. A kit for a process vessel, comprising:

a bag including a neck;
a connector defining an aperture and including a welding surface surrounding the aperture, wherein the welding surface is configured to be heat-sealed to the neck of the bioreactor bag; and
an impeller assembly including an impeller shaft and impeller blade configured to pass through the aperture of the connector,
wherein the impeller assembly and the connector are configured to be mechanically joined to one another.

Aspect 41. The kit according to aspect 40, further comprising a clamp, and wherein:

the connector includes an outwardly extending flange,
the impeller assembly includes an impeller flange configured to contact the outwardly extending flange of the connector, and
the impeller assembly and the connector are configured to be mechanically joined to one another by using the clamp to secure the outwardly extending flange of the connector and the impeller flange to one another.

Aspect 42. The kit according to aspect 40, wherein the impeller assembly and the connector are configured to be mechanically joined to one another by a press-fit between the impeller assembly and a cup formed at an end of the connector.

Aspect 43. The kit according to aspect 40, wherein the impeller assembly and the connector are configured to be mechanically joined to one another by engagement features included on at least one of the impeller assembly or the connector.

Aspect 44. The kit according to aspect 40, wherein the impeller assembly and the connector are configured to be mechanically joined to one another by engagement of a captive nut disposed on one of the connector or the impeller assembly engaging with threading provided on the other of the connector or the impeller assembly.

Aspect 45. The kit according to aspect 40, wherein the impeller assembly and the connector are configured to be mechanically joined to one another by a plurality rotating bolts and locking nuts included in one of the impeller assembly or the connector, and a plurality of recesses configured to receive the rotating bolts formed in the other of the impeller assembly or the connector.

Aspect 46. The kit according to any of aspects 40-45, wherein when the impeller assembly and the connector are mechanically joined to one another, a seal is formed between the impeller assembly and the connector.

Aspect 47. The kit according to any of aspects 40-46, wherein the bag and the connector each comprise a polyolefin.

Aspect 48. The kit according to any of aspects 40-47, wherein the bag and the connector each comprise a fluoropolymer.

Aspect 49. The kit according to any of aspects 40-48, wherein the welding surface is defined on an inner surface of the connector.

Aspect 50. The kit according to any of aspects 40-49, wherein the welding surface is defined on an outer surface of the connector.

Aspect 51. The kit according to any of aspects 40-50, wherein the bag is a gusseted three dimensional bag.

Aspect 52. The kit according to any of aspects 40-51, wherein the process vessel is a bioreactor and the bag is a bioreactor bag.

Aspect 53. A method of assembling a process vessel, comprising:

bonding a neck of a bag to a welding surface of a connector, the connector defining an aperture;
inserting a blade and a shaft of an impeller assembly through the aperture of the connector into the bag; and
mechanically fixing the impeller assembly to the connector.

Aspect 54. The method according to aspect 53, wherein mechanically fixing the impeller assembly to the connector includes clamping an impeller flange included in the impeller assembly to a connector flange included in the connector.

Aspect 55. The method according to aspect 53, wherein mechanically fixing the impeller assembly to the connector includes pressing the connector and the impeller assembly together to form a press-fit.

Aspect 56. The method according to aspect 53, wherein mechanically fixing the impeller assembly to the connector includes bringing engagement features included on one of the impeller assembly or the connector into contact with the other of the impeller assembly or the connector.

Aspect 57. The method according to aspect 53, wherein mechanically fixing the impeller assembly to the connector includes engaging a captive nut disposed on one of the impeller assembly or the connector with threading provided on the other of the impeller assembly or the connector.

Aspect 58. The method according to any of aspects 53-57, wherein the bonding includes heat welding.

Aspect 59. The method according to any of aspects 53-58, wherein the process vessel is a bioreactor and the bag is a bioreactor bag.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. A kit for a process vessel, comprising:
a bag including a neck;
a connector defining an aperture and including a welding surface surrounding the aperture, wherein the welding surface is configured to be heat-sealed to the neck of the bioreactor bag; and
an impeller assembly including an impeller shaft and impeller blade configured to pass through the aperture of the connector,
wherein the impeller assembly and the connector are configured to be mechanically joined to one another.

2. The kit of claim 1, further comprising a clamp, and wherein:
the connector includes an outwardly extending flange,
the impeller assembly includes an impeller flange configured to contact the outwardly extending flange of the connector, and
the impeller assembly and the connector are configured to be mechanically joined to one another by using the clamp to secure the outwardly extending flange of the connector and the impeller flange to one another.

3. The kit of claim 1, wherein the impeller assembly and the connector are configured to be mechanically joined to one another by a press-fit between the impeller assembly and a cup formed at an end of the connector.

4. The kit of claim 1, wherein the impeller assembly and the connector are configured to be mechanically joined to one another by engagement features included on at least one of the impeller assembly or the connector.

5. The kit of claim 1, wherein the impeller assembly and the connector are configured to be mechanically joined to one another by engagement of a captive nut disposed on one of the connector or the impeller assembly engaging with threading provided on the other of the connector or the impeller assembly.

6. The kit of claim 1, wherein the impeller assembly and the connector are configured to be mechanically joined to one another by a plurality rotating bolts and locking nuts included in one of the impeller assembly or the connector, and a plurality of recesses configured to receive the rotating bolts formed in the other of the impeller assembly or the connector.

7. The kit of claim 1, wherein when the impeller assembly and the connector are mechanically joined to one another, a seal is formed between the impeller assembly and the connector.

8. The kit of claim 1, wherein the bag and the connector each comprise a polyolefin.

9. The kit of claim 1, wherein the bag and the connector each comprise a fluoropolymer.

10. The kit of claim 1, wherein the welding surface is defined on an inner surface of the connector.

11. The kit of claim 1, wherein the welding surface is defined on an outer surface of the connector.

12. The kit of claim 1, wherein the bag is a gusseted three dimensional bag.

13. The kit of claim 1, wherein the process vessel is a bioreactor and the bag is a bioreactor bag.

14. A method of assembling a process vessel, comprising:
bonding a neck of a bag to a welding surface of a connector, the connector defining an aperture;
inserting a blade and a shaft of an impeller assembly through the aperture of the connector into the bag; and
mechanically fixing the impeller assembly to the connector.

15. The method of claim 14, wherein mechanically fixing the impeller assembly to the connector includes clamping an impeller flange included in the impeller assembly to a connector flange included in the connector.

16. The method of claim 14, wherein mechanically fixing the impeller assembly to the connector includes pressing the connector and the impeller assembly together to form a press-fit.

17. The method of claim 14, wherein mechanically fixing the impeller assembly to the connector includes bringing engagement features included on one of the impeller assembly or the connector into contact with the other of the impeller assembly or the connector.

18. The method of claim 14, wherein mechanically fixing the impeller assembly to the connector includes engaging a captive nut disposed on one of the impeller assembly or the connector with threading provided on the other of the impeller assembly or the connector.

19. The method of claim 14, wherein the bonding includes heat welding.

20. The method of claim 14, wherein the process vessel is a bioreactor and the bag is a bioreactor bag.
